(19) United States
(12) Patent Application Publication
(10) Pub. No.: US 2004/0005640 A1
(43) Pub. Date: Jan. 8, 2004

(54) Assay for transformed alpha fetal protein

(76) Inventor: Gerald J. Mizejewski, Clifton Park, NY (US)

Correspondence Address: MICHAEL P. WILLIAMS, BOND, SCHOENECK & KING, PLLC, ONE LINCOLN CENTER, SYRACUSE, NY, 13202, US

(21) Appl. No.: 10/424,018

(22) Filed: Apr. 25, 2003

Related U.S. Application Data
(60) Provisional application No. 60/375,576, filed on Apr. 25, 2002.

(51) Int. Cl. G01N 33/53

(52) U.S. Cl. 435/7.9

(57) ABSTRACT

A method for determining the existence of transformed alpha fetal protein in a sample. The first step is mixing said reagents by inversion before use. The second step is providing a microtiter plate with wells coated with synthetic TAFP diluted in a coating buffer, dispensing PSB buffer to wet the well surfaces, waiting a period of time and decanting the microtiter plate. The third step is providing a first washing of the well(s) with a wash buffer for removing unbound material. The fourth step is adding non-fat dry milk to the coating buffer and incubating the microtiter plate for a first period of time at room temperature for blocking nonspecific binding sites. The fifth step is providing a second washing of the well with a wash buffer for removing unbound material. The sixth step is adding anti-TAFP antibody diluted in a binding buffer for a second period of time for allowing the primary antipetide antibodies to bind. The seventh step is providing a third washing of the well(s) with a wash buffer for removing unbound material. The eighth step is adding diluted goat anti-rabbit immunoglobuin-horseradish peroxidase conjugate in TBST for binding of the primary to the secondary antibody and then incubating for a third period of time. The ninth step is providing a fourth washing of the well(s) with a TBST-containing buffer for removing unbound material. The tenth step is adding horseradish peroxidase substrate and incubating at room temperature for developing color. The eleventh step is adding stop solution to said well after a fourth period of time for stopping the test. Finally, the twelfth step is determining the absorbance of the alpha fetal protein peptide at a wavelength using a microplate reader.

Priority is claimed to provisional application U.S. Patent Application No. 60/375,576 entitled “Diagnostic Assay for Tumors Using Alpha Fetal Protein Peptide Fragments,” filed on Apr. 25, 2002, the entire disclosure of which is hereby incorporated by reference.

### FIELD OF THE INVENTION

The present invention relates to an assay method to determine the presence of transformed alpha fetal protein.

### BACKGROUND OF THE INVENTION

Many biochemical techniques are available to test for the presence of proteins or peptides including antibody detection techniques such as immunodiffusion, serum neutralization, immunofluorescent staining and the enzyme-linked immunosorbent assay (ELISA). The samples for these tests are usually blood, plasma, serum or other body fluids or tissues. All of these tests involve the binding of antibodies in the sample with controlled amounts of antigen. The antigen may either be added in liquid form, as is done with immunodiffusion plates, or immobilized on a surface as part of a test system, as is done with the ELISA technique. Detection of antibody is done by viewing a precipitation line in immunodiffusion techniques and by observing color generation in enzymatic techniques.

The nucleotide and amino acid sequences of human alpha-fetoprotein (AFP or HAFP) are known. AFP is a glycoprotein that is produced during gestation, initially by the fetal yolk sac and then in the fetal liver. Furthermore, AFP is a major serum protein constituent of the fetal plasma throughout the gestation period. However, upon partition the gene for AFP is repressed and its serum concentration diminishes to a negligible level.

Studies have shown that when AFP is incubated with a molar excess of the ligand estradiol, the AFP undergoes a change in conformation. This conformational change results in a transformed alpha-fetoprotein (“TAFP”). It has been discovered that the presence of TAFP in blood or other tissue can act as a marker for steroid stimulated tissues including estrogen-stimulated breast cancer cells and liver cancer cells. In addition, the presence of TAFP in amniotic fluid or maternal sera acts as a marker for fetal defects, specifically defects related to growth restriction/retardation. Examples of such defects include neural tube defects, Down Syndrome, Smith-Lemnli-Optiz Syndrome and anencephaly. In addition, the presence of TAFP can act as a pregnancy test for non-primate mammals. The current pregnancy tests for non-primate mammals include steroidal based tests and placental lactogen tests which both lack specificity. Non-primate mammals lack the pregnancy related protein human chorionic gonodotrophin (HCG), which is the reagent used in human pregnancy tests. Since non-primates lack HCG, there is not currently available a suitable pregnancy test for non-primate mammals. Data has shown that the present invention can be employed as both a pregnancy test and a marker of fetal defects, distress, and viability in non-primate mammals.

What is needed is a diagnostic test method and apparatus that can determine the presence of transformed alpha-fetoprotein fragments that is simple to use, economical, rapid and require no special equipment.

### SUMMARY OF THE INVENTION

It is an aspect of the present invention to provide a diagnostic assay for determining information relating to the presence of TAFP in a blood or tissue sample.

It is another aspect of the present invention to provide an economical and simple to use diagnostic assay and diagnostic assay kit for determining the presence of TAFP in a blood or tissue sample.

To accomplish these and other aspects of the invention a method for determining the existence of transformed alpha fetal protein includes several steps. The first step is mixing said reagents by inversion. The second step is providing a microtiter plate with wells coated with synthetic TAFP diluted in a coating buffer, dispensing PSB buffer to wet the well surfaces, waiting a period of time and decanting the microtiter plate. The third step is providing a first washing of the well(s) with a wash buffer for removing unbound material. The fourth step is adding non-fat dry milk to the coating buffer and incubating the microtiter plate for a first period of time at room temperature for blocking nonspecific binding sites. The fifth step is providing a second washing of the well with a wash buffer for removing unbound material. The sixth step is adding anti-TAFP antibody diluted in a binding buffer for a second period of time for allowing the primary antipetide antibodies to bind. The seventh step is providing a third washing of the well(s) with a wash buffer for removing unbound material. The eighth step is adding diluted goat anti-rabbit immunoglobuin-horseradish peroxidase conjugate in TBST for binding of the primary to the secondary antibody and then incubating for a third period of time. The ninth step is providing a fourth washing of the well(s) with a TBST-containing buffer for removing unbound material. The tenth step is adding horseradish peroxidase substrate and incubating at room temperature for developing color. The eleventh step is adding stop solution to said well after a fourth period of time for stopping the test. Finally, the twelfth step is determining the absorbance of the alpha fetal protein peptide at a wavelength using a microplate reader.

An apparatus for determining the existence of tumors using alpha fetal protein peptide fragments includes an anti-TAFP coated microtiter plate for conducting a correct test; color-coded reagents for determining test standards, test controls; assay buffer; wash concentrate; horseradish peroxidate substrate, and; stop solution for stopping the test.

These and other aspects of the invention will become apparent from the following description, the description being used to illustrate the preferred embodiment of the invention.

### DETAILED DESCRIPTION OF THE INVENTION

Some of the most potent growth inhibitors are peptide fragments derived from abundant plasma or structural proteins that do not inhibit growth. The internal segments that comprise a class of growth regulators consisting of fragments of abundant body proteins appear to be a recurring theme in the field of growth regulation. For example, the mammalian alpha-fetoprotein, or alpha fetal protein, (AFP) is a tumor-associated fetal protein synthesized mostly in fetal liver and hepatomas. The AFP comprises a molecular mass of 69,000 Daltons consisting largely of an alpha-helical molecule of three major domains. The AFP in its native form displays mostly growth enhancing properties consistent with its role as a major serum transport protein of the fetus. It was recently discovered than an amino acid segment that can assume a beta sheet/turn conformation in the third domain of human AFP can serve as a growth inhibitor motif. This peptide region encompasses a sequence stretch of 34-amino acids and is buried in a molecular cleft in the naturally-folded form of AFP. Consequently, this epitope cannot be detected using present-day conventional and/or commercial antisera directed against human alpha-fetoprotein (HAFP). This peptide was initially discovered by sequence similarity with members of the heat shock protein family (HSP-70 and HSP-90) that are associated with steroid receptor binding and protein folding in the endoplasmic reticulum.

In U.S. Pat. No. 5,674,842, which is incorporated herein by reference, a synthetic form of the 34 mer amino acid segment termed growth-inhibitory-peptide (GIP) is described. Applicants have produced an antiserum using methods well known in the art against the GIP segment which is exposed on the partially unfolded form of the AFP molecule, or transformed AFP (TAFP). This antiserum constitutes the basis of the present invention ELISA platform. Upon conformational change (tertiary structure) of HAFP to TAFP, the GIP segment is exposed and can be quantified by conventional immunoassay methodology. Furthermore, the epitope is a shock/stress-induced site that is revealed by subjection of native HAFP to ionic, pH, osmotic shock or anoxia, ischemia and high ligand concentration environments. The GIP can also be detected following in vitro conditions using urea, guandidine-HCl, pH, temperature extremes and alcohols. In addition, this site can be unmasked following exposure to high ligand concentrations of steroids, fatty acids, growth factors and peptic hormones. For example, during pregnancy in a certain percentage of HAFP molecules the GIP is exposed following fetal physiochemical insult and remains as such until gestational homeostasis is re-established. The HAFP is largely a growth enhancing protein since most fetal growth continues unabated in the presence of the native AFP. Therefore, a growth inhibitor site on the native HAFP would normally be concealed or hidden in order for the normal growth to proceed. However, at times during normal growth it is expected that growth inhibition might serve a physiological purpose during the fetal organ/tissue construction (histogenesis and organogenesis), in neoplastic growth and differentiation.

The method for determining the existence of TAFP uses a two-tier antibody competitive (indirect) immunoabsorbant (ELISA) microtiter plate assay. The analyte (TAFP) to be measured in solution competes with a TAFP peptide absorbed on the well of a microtiter plate for binding to a polyclonal rabbit antiserum directed against the TAFP epitope. Following the competition, the antibodies that remain bound to the microtiter wall are detected by horseradish peroxidase-labeled goat anti-rabbit IgG. An appropriate substrate as is known in the art is provided for enzyme cleavage that produces a brownish color signal. The standards and controls procedures are accomplished similarly. The unbound materials are removed by decanting and washing the microtiter plates.

In the preferred embodiment of the invention the TAFP coated-well ELISA contains sufficient color-coded reagents for 96 determinations, which include standards, controls and unknown specimens. The apparatus test kit for determining the existence of TAFP includes TAFP standard's reagents, anti-TAFP reagent, anti-TAFP coated microtiter plate, TAFP control's reagents, assay buffer, wash concentrate, horseradish peroxidase substrate and stop solution.

The TAFP standard's reagents include one vial (5 mLs) labeled A contains 0.0 ng/ml of TAFP. There are five vials (0.5 ml each) labeled B through F containing lyophilized TAFP concentration of about 5, 20, 100, 200 and 500 ng/ml TAFP in a protein-based buffer with a non-mercury preservative. To accomplish a correct test dilutents are added to the vials to solubilize the reagents. Appropriately, each vial contains a label that describes the exact concentrations of dilutents required. These reagents are stored as liquids at about 2° C. to 8° C. for only up to 24 hours. For longer periods these reagents are stored as lyophilized kit reagents at about 4° C. until their expiration date.

The anti-TAFP reagent includes one bottle (25 ml) containing the primary antibody (rabbit anti-TAFP epitope) and is supplied in a protein-based buffer with sodium azide as a preservative. The color of this reagent is yellow and the reagent is stored at about 2° C. to 8° C. until its expiration date.

The anti-TAFP coated microtiter plate contains 96-wells in the preferred embodiment of the invention. However, a practitioner in the art readily understands the 96-well microtiter plate is substitutable by a microtiter plate containing a differing number of wells The number of wells depend upon the application and testing methods. The microtiter plate is coated with a predetermined amount of TAFP to bind the primary antibody. All the wells of the microtiter plate have been coated with TAFP. The microtiter plate is stored at about 2° C. to 8° C. until its expiration date.

The TAFP control's reagents include three vials (0.5 mls each) containing Levels I, II and III that, respectively, represent low, medium and high range concentrations of TAFP in a protein-based buffer with a non-mercury preservative. These reagents contain labels on the vials describing control ranges. These reagents are stored as a liquid at about 2° C. to 8° C. for up to two weeks. The unused reagents are stored at about 4° C. until their expiration date.

The assay buffer includes one bottle (25 mls) containing a protein-based buffer with a non-mercury preservative. The assay buffer is stored at about 2° C. to 8° C. until its expiration date.

The wash concentrate includes one bottle (60 mls) containing buffered saline with a non-ionic detergent. The wash concentrate is diluted 25-fold with deionized water prior to its use. The wash concentrate is stored at about 2° C. to 8° C. until its expiration date.

The horseradish peroxidase substrate includes one vial (25 mls) containing the horseradish peroxidase substrate. The horseradish peroxidase substrate is diluted 25-fold with deionized water prior to its use. The horseradish peroxidase substrate is stored at about 2° C. to 8° C. until its expiration date.

The stop solution includes one bottle (25 mls) contain the enzyme stop solution. The stop solution is stored at about 2° C. to 8° C. until its expiration date.

Some of the reagents in the apparatus (kit) for determining the existence of TAFP contain sodium azide as a preservative. For all such reagents, the concentration of sodium azide is ≦0.09%.

The serum should be used and the usual precautions for venipuncture should be observed. The specimens may be stored at about 2° C. to 8° C. for up to 24 hours and frozen at about −20° C. or lower for longer periods. The repeated freezing and thawing of the samples should be avoided. Furthermore, grossly hemolyzed or lipemic specimens should not be used. The frozen samples should be thawed and mixed thoroughly by gentle swirling or inversion prior to use.

After removing the reagents from the refrigerator they shall be allowed to attain room temperature (about 25° C.) before pipetting. The standards and controls should be mixed before use by inverting or swirling gently rather than vortexing. To insure homogeneous mixture of the final reagents in each assay tube a thorough shaking or gentle vortexing may be required. Furthermore, failure to obtain the appropriate values for controls may indicate imprecise manipulations, improper sample handling or deterioration of reagents. The failure to blot tubes adequately following decantation may result in poor replication and spurious values.

The method for determining the existence of TAFP first allows all reagents to reach room temperature (about 25° C.) and they are mixed thoroughly by inversion before use. The assay standards, controls and unknowns are run in duplication. Next, a 96-well microtiter plate has been coated with 100 μl of synthetic TAFP peptide (2 nmol/ml) diluted in a coating buffer. About 200 μl of PBS buffer are dispensed to wet the well surfaces. The microtiter plated is decanted after a 5-minute wait. After this wait the microtiter wells are provided a first washing 3-times with 200 μl aliquots of wash buffer. One skilled in the art would recognize that various wash buffers can be utilized in this and subsequent wash step, such as a phosphate buffered saline with Tween-20. The next step is to add 200 μl of 5% non-fat dry milk in buffer and incubate for a first period of time at room temperature to block nonspecific binding sites. The first period of time is about one hour at a room temperature of about 25° C. The microtiter wells are provided a second washing 3-times with 200 μl aliquots of wash buffer.

The method for determining the existence of TAFP continues by adding 100 μl of anti-TAFP antibody diluted in a binding buffer for a second period of time at a specific temperature to allow the primary antipeptide antibodies to bind. The second period of time is for two hours at 37° C. or for 16 hours at 4° C. The microtiter wells are provided a third washing 3-times with 200 μl aliquots of wash buffer. In proceeding with this method 100 μl of diluted goat anti-rabbit immunoglobuin-horseradish peroxidase conjugate in TBST is added to allow binding of the primary to the secondary antibody. This is then incubated for a third period of time at room temperature. The third period of time is about two hours at 25° C. The microtiter wells are provided a fourth washing 4 to 5-times with TBST-containing buffer.

The final steps consists of adding 100 μl of horseradish peroxidase substrate and incubating at room temperature until color develops. After a fourth period of time which is usually about 30 minutes, but may be any predetermined time required to generate a correct test result, 100 μl of stop solution, typically a 2M sulfuric acid, is added to each well. This is to bring the reactions in the assay to a halt so that measurements can take place. Finally, the absorbance is determined by using a microplate reader. The absorbency is determined at any appropriate wavelength and in the preferred embodiment is approximately 410 nm.

The results of the method for determining the existence of TAFP fragments are typically calculated using a log-log curve fit. The plot curve is of the net cpm or % B/T (y axis) against the TAFP concentration on the log-log graph. Alternately, reduction methods typically give slightly different results. The absorbance is calculated by subtracting the mean absorbance of the 0 ng/mL AFP standard from the mean standards, controls and unknowns. The % B/T is calculated for each standard and unknown as follows:

%B/T=Net Absorbance/Mean Absorbance×100

The concentrations of the net absorbance or % B/T for each control and unknown are determined from the log-log curve. Any sample reading greater than the highest standard should be appropriately compared with the 0 ng/mL AFP standard and reassayed. Finally, any sample reading lower than the lowest standard should be reported as zero. However, the reagents supplied in the kit are optimized to measure TAFP levels in sera.

The transformed alpha-fetoprotein (TAFP) ELISA coated-well kit provides materials for the quantitative measurement of conformationally-altered AFP in biological fluids including but not limited to serum, plasma, urine, nipple apirates and amniotic fluid. It is intended strictly for in vitro use and can be used as a marker to aid in the diagnosis, screening and monitoring of various human neoplastic and benign growth disorders such as steroid stimulated tissues including estrogen-stimulated breast cancer cells and liver cancer cells.

The TAFP assay does not detect any specific congenital malformation as do present day AFP assays, but a state of fetal growth restriction/retardation resulting from physicochemical fetal insults. For example, both neural tube defects (high AFP) and Down Syndrome (low AFP) produce growth retarded fetuses and are detected in the TAFP assay. Furthermore, the GIP is not exposed (or is to a lesser extent) in normal cord sera, maternal sera and amniotic fluid. The ancillary detection of other fetal adverse outcomes that accompany third trimester growth retardation are an added bonus and might include pre-term delivery, low birth weight, small for gestational age and fetal demise. Consequently, an important use of early onset growth restriction might be as a predictor of severe pre-eclampsia in later gestation (third trimester). The TAFP assay could be implemented into the routine prenatal screening (second trimester) of 15 to 20 weeks gestation for assessment of growth restriction, growth retardation and other congenital anomalies. For example, the congenital anomalies could include chromosomal abnormalities and anatomic structural defects including neural tube defects, Down Syndrome, Smith-Lemnli-Optiz Syndrome and anencephaly.

### EXAMPLE 1

Example 1

Human pregnancy samples of maternal serum, and amniotic fluid were assayed for the presence of transformed (T) alpha-fetoprotein (AFP) by an ELISA microtiter kit. ‡ The immunoassay employed an antibody to an occult epitope on the third domain of AFP. Values are in nanogram or microgram/mL AFP.

| Pregnancy Condition | Pregnancy | Maternal Sera | Amniotic Fluid |
|---|---|---|---|
| Assayed | Trimester | (Ng/mL) | (μg/ml) |
| 1) Normal | First | 0.56 | 0.10 |
| (Non-affected) | | (5) | |
| | Second | 0.13 | |
| | | (6) | |
| 2) Down Syndrome | First | 10.3* | 1.8 |
| | | (3) | (2) |
| | Second | 0.40 | |
| | | (3) | |
| 3) Neural Tub | Second | 8.00* | 3.1 |
| Defects | | (3) | (3) |
| 4) Smith-Lemuli- | Second | 30** | |
| Opitz Syndrome | | (8) | |
| (Growth Retardation) | | | |

*Note that these values are >5-fold that of control.
**Note that these values are >200-fold that of control. Neural tube defects include spina bifida, anencephaly & hydrocoele

In addition, the presence of TAFP can act as a pregnancy test for non-primate mammals. Data has shown that the present invention can be employed as both a pregnancy test and a marker of fetal defects, distress, and viability in non-primate mammals.

### EXAMPLE 2

Example 2

Presence of the Transformed (T) alpha-fetoprotein (AFP) epitope in pregnant versus non-pregnant mammals of non-primate origin. Biological fluids containing the TAFP were quantitated using a ELISA kit *specific for the AFP-derived third domain epitope. Fluids included maternal serum and urine specimens**. Mean Ng/ml values are listed

Type of Sample

| Maternal Sera | | Urines | | | | | | Maternal Sera | |
|---|---|---|---|---|---|---|---|---|---|
| Asian Benteng Cows | | Brown Bears | | Polar Bears | | Panda Bears | | Elephants | |
| Non- | | Non- | | Non- | | Non- | | Non- | |
| Pregnant | Pregnant | Pregnant | Pregnant | Pregnant | Pregnant | Pregnant | Pregnant | Pregnant | Pregnant |
| N = 3 | N = 3 | N = 2 | N = 2 | N = 2 | N = 3 | N = 5 | N = 3 | N = 2 | N = 8 |
| Mean < 25.0 | 150 | 1.2 | 250 | 1.2 | 100 | 12 | 450 | 900 | 800 |
| Range 10-25 | 50-175 | 0.5-2 | 200-300 | 0.5-2 | 90-110 | 3-20 | 300-550 | 850, 950 | 200-600 |

What is claimed is:

1. A method for determining the existence of transformed alpha fetal protein in a sample, said method comprising the steps of:
a) providing a microtiter plate with wells coated with synthetic TAFP diluted in a coating buffer, dispensing PSB buffer to wet said well surfaces, waiting a period of time and decanting said plate;
b) washing said well with a wash buffer for removing unbound material;
c) adding non-fat dry milk to said coating buffer and incubating said microtiter plate for a first period of time;
d) washing said well with a wash buffer for removing unbound material;
e) adding anti-TAFP antibody diluted in a binding buffer for a second period of time for allowing the primary antipetide antibodies to bind;
f) washing said well with a wash buffer for removing unbound material;
g) adding diluted goat anti-rabbit immunoglobuin-horseradish peroxidase conjugate in TBST for binding of the primary to the secondary antibody and then incubating for a third period of time;
h) washing said well with a TBST-containing buffer for removing unbound material;
i) adding horseradish peroxidase substrate and incubating to develop color;
j) adding stop solution to said well for stopping the test; and
k) determining the absorbance at a selected wavelength.

2. The method of claim 1, wherein said wavelength is approximately 410 nm.

3. An apparatus for determining the existence transformed alpha fetal protein comprising:
an anti-TAFP coated plate;
color-coded reagents for determining test standards;
test controls;
an assay buffer;
a wash concentrate;
a horseradish peroxidase substrate; and,
a stop solution for stopping the test.
